(19) United States
(12) Patent Application Publication
(10) Pub. No.: US 2006/0269504 A1
(43) Pub. Date: Nov. 30, 2006

(54) Use of alkyl resorcinols in the treatment of acne

(76) Inventor: Alexander Gordon James, Shambrook (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/553,641

(22) PCT Filed: Dec. 19, 2003
(86) PCT No.: PCT/EP03/14866

§ 371 Date: Oct. 14, 2005

(30) Foreign Application Priority Data
Apr. 15, 2003 (EP) 0325390.4

(51) Int. Cl. A61K 31/22; A61K 31/05; A61K 31/075; A61K 8/35

(52) U.S. Cl. 424/70.31; 514/546; 514/718; 514/734

(57) ABSTRACT

An anti-acne composition for topical application comprises: (i) from 0.01% to 20% by weight of a compound of formula (I), wherein: R1, R2, R3 and R4 are the same or different and are selected from H, OH, and OCH3; and R is alkyl containing from 1 to 20 carbon atoms; the compound of formula (I) optionally being in the form of an ester with a carboxylic acid comprising an alkyl group containing from 1 to 6 carbon atoms; (ii) between 0.1% and 20% by weight of at least one further anti-acne agent; (iii) from 0% to 30% by weight of at least one surfactant; and (iv) topically acceptable diluent or carrier.

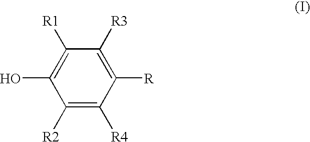

This invention relates to compositions for the treatment of acne, to methods of treating acne using the compositions and to the use of certain compounds in the treatment of acne.

Acne is a common inflammatory pilosebaceous disease characterized by comedones, papules, pustules, inflamed nodules, superficial pus-filled cysts, and in extreme cases, sinus formation and deep inflammation, sometimes associated with purulent sacs. For example, acne vulgaris is a polymorphic skin eruption characterised clinically by blackheads, white heads, papules, nodules, cysts and scars occurring particularly on areas of the skin rich in sebaceous glands, such as the face, forehead and back.

The pathogenesis of acne is complex. An interaction between hormones, keratinization, sebum, and bacteria somehow determines the course and severity of the disease. Acne begins at puberty when the increase of androgens causes an increase in the size and activity of the sebaceous glands. The earliest microscopic change is intrafollicular hyperkeratosis, which leads to restriction of the pilosebaceous follicle with consequent formation of the comedone composed of sebum, keratin, and microorganisms, particularly Propionibacterium acnes. Lipases from P. acnes break down triglycerides in the sebum to form free fatty acids (FFA), which may irritate the follicular wall. Retention of sebaceous secretions and dilation of the follicle may lead to cyst formation. Rupture of the follicle with release of the contents into the tissues induces an inflammatory reaction which heals with scarring in severe cases.

Acne tends to appear during puberty and to fade away again, usually spontaneously when growth has stopped. Only rarely does it recede before the age of 20 and occasionally it is still to be found at the age of 30 and beyond. The face, back, and shoulders are the predominant areas affected.

Acne can be treated by topical application of various lotions, salves, and the like or by, for example, localised treatment with sulphur, resorcinol, salicylic acid, benzoyl peroxide, vitamin A acids, antibiotics such as erythromycin, and the like.

U.S. Pat. No. 5,416,075 discloses the use of 4-hexylresorcinol as an antimicrobial compound in an oral hygiene composition. Although the document has a section that describes anti-acne agents and although this section includes resorcinol and resorcinol monoacetate, there is no mention of alkyl resorcinols in this context.

Hexylresorcinol is categorized as an antibacterial agent used for treating skin infections, for treating skin wounds and as a wound cleanser in U.S. Pat. No. 4,895,727. Acne treatment compounds are also described in this document, but no alkyl resorcinol compounds are mentioned in this category of compounds, nor are alkyl resorcinols suggested as having any anti-acne activity.

U.S. Pat. No. 3,137,622 relates to compositions for topical application to the skin in the treatment of acne. The compositions contain a hydroxyaromatic compound that has a keratolytic or keratoplastic effect. The hydroxyaromatic compounds specifically mentioned are resorcinol, resorcinol monoacetate, hexylresorcinol, cresol and metacresyl acetate. Resorcinol is used in the examples, and resorcinol and resorcinol monoacetate are said to be particularly preferred. No examples are given of a composition containing hexylresorcinol. The hydroxyaromatic compounds are used as the sole keratolytic agent and another compound, such as hexachlorophene, is required to be present in the compositions in order to provide antibacterial activity. There is no suggestion in the document that the hydroxyaromatic compounds could have an antibacterial effect or that they could themselves be used as the antibacterial agent in the composition.

Bacteria that are present in or on the skin can have positive or negative physiological effects, depending on the particular bacterium. There can therefore be benefits in selectively targeting the bacteria having negative effects, such as P. acnes.

There remains a need for compounds and compositions that are effective in the treatment of acne. Compounds that are capable of being delivered effectively to the target site and have selective antibacterial effects, and compositions that have beneficial secondary effects, are particularly desirable.

According to the present invention, there is provided an anti-acne composition for topical application comprising:
(i) from 0.01% to 20% by weight of a compound of formula (I) 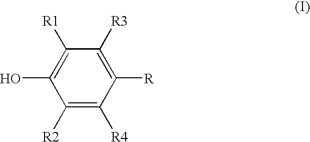
   wherein:
   R1, R2, R3 and R4 are the same or different and are selected from H, OH, and OCH₃; and
   R is alkyl containing from 1 to 20 carbon atoms;
   the compound of formula (I) optionally being in the form of an ester with a carboxylic acid comprising an alkyl group containing from 1 to 6 carbon atoms;
(ii) between 0.1% and 20% by weight of at least one further anti-acne agent;
(iii) from 0% to 30% by weight of at least one surfactant; and
(iv) a topically acceptable diluent or carrier.

In another aspect, the present invention provides the use of the composition of the invention in the manufacture of a medicament for the treatment of acne.

A further aspect of the invention is a method of treating acne in humans which comprises topically applying to the skin of a human suffering from acne a composition of the invention. The method can be therapeutic or cosmetic, but will generally be therapeutic.

The present invention is based on the surprising finding that compounds of formula (I) are particularly effective as antibacterial agents in the treatment of acne. Thus, the compounds of formula (I) are unexpectedly effective antibacterial agents against Propionibacterium species, for example P. acnes, compared to other bacteria present on the skin and should have good penetration into the skin. This selective antibacterial effect was particularly surprising. Moreover, there can be advantages when the compounds of formula (I) are used as the antibacterial agent together with further anti-acne agents. This is particularly the case when the further anti-acne agent is selected from desquamators, keratolytics, comedolytics and exfoliants. In these situations, there is an advantage from the combination of at least two therapeutic ingredients with distinct, but complementary, modes of action. An advantage is also gained from the enhanced delivery of the antibacterial agent of formula (I), due to increased skin and follicular penetration as a consequence of the desquamating, keratolytic, comedolytic or exfoliating action of the further anti-acne agent. A further advantage is gained due to the selective antibacterial effect of the compound of formula (I) against the organisms implicated in acne vulgaris, typically Propionibacterium species, especially P. acnes.

The compositions of the invention may comprise a single further anti-acne agent or a mixture of two or more anti-acne agents.

For the compounds of formula (I), R1, R2, R3 and R4 are preferably selected from H and OH, and more preferably at least two of R1, R2, R3 and R4 are OH. R is preferably alkyl containing from 4 to 14, more preferably 5 to 8 carbon atoms.

The term alkyl, as used herein, refers to straight chain and branched aliphatic and alicyclic saturated hydrocarbon groups (preferably the alkyl groups are aliphatic). Alkyl groups may contain one or more carbon-carbon double bonds, but are preferably saturated. Non-limiting examples of alkyl groups are methyl, ethyl, propyl, butyl, isopropyl, isobutyl, pentyl, hexyl, heptyl, octyl, 2-ethylhexyl, cyclopentyl, cyclohexyl, nonyl, decyl, undecyl, dodecyl, tridecyl and tetradecyl.

The compound of formula (I) is optionally in the form of an ester with a carboxylic acid comprising an alkyl group containing from 1 to 6 carbon atoms. Esters may be formed with one or more or all of the OH groups in the compound of formula (I). Suitable carboxylic acids include acetic acid, propionic acid and butyric acid; of these, acetic acid is preferred. The compounds may therefore be, for example, mono- or di-acetates.

Preferred compounds of formula (I) have the formula (II) 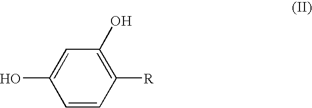
wherein R is alkyl containing from 1 to 20 carbon atoms, such as from 4 to 14 carbon atoms, more preferably 5 to 8 carbon atoms.

An example of a preferred compound of formula (I) is 4-hexylresorcinol. 4-Hexylresorcinol has been found to be particularly effective as an anti-acne agent.

Other examples of compounds of formula (I) are 4-pentylresorcinol, 4-hexylresorcinol monoacetate, 4-hexylresorcinol diacetate, 4-heptylresorcinol, 4-octylresorcinol and 4-nonylresorcinol.

The compounds of formula (I) can be used in the present invention either singly or as mixtures of different compounds of formula (I).

The compositions of the invention comprise the one or more compounds of formula (I) in an amount of from 0.01% to 20% by weight, such as from 0.1% to 10% by weight, preferably from 0.6% to 5% by weight, more preferably from 0.8% to 3% by weight, e.g., from 1.5% to 2.5% by weight. Amounts by weight are based on the total weight of the composition.

Topically-Acceptable Diluents and Carriers

The compositions of the invention comprise a topically acceptable diluent or carrier (i.e., a dermatologically/cosmetically acceptable vehicle) to act as a diluent, dispersant or carrier for the active. The carrier may comprise materials commonly employed in skin care products such as water, liquid or solid emollients, silicone oils, emulsifiers, solvents, humectants, thickeners, powders, propellants and the like, some of which are also described separately herein under specific headings.

The compositions that are useful in the present invention comprise a safe and effective amount of the topically-acceptable carrier or diluent which can have a variety of different forms. By “safe and effective” is meant an amount sufficient to act as a suitable vehicle for the required components and any other optional components, but not so much as to cause any side effects or skin reactions. The topically-acceptable carrier should be non-irritant. “Topically-acceptable” therefore means that the carrier is suitable for topical application to the skin without causing any untoward safety or toxicity concerns. In other words, these carriers are suitable for use on mammalian skin. The typical carrier can be in the form of a hydroalcoholic system (e.g. liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems. Nonlimiting examples of the topical carrier systems useful in the present invention are described in the following four references, all of which are incorporated herein by reference in their entirety: “Sun Products Formulary”, Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3, 1981.

The topically-acceptable diluents or carriers, in total, typically constitute from about 0.1% to about 99.8% by weight of the compositions of the present invention, preferably from about 80% to about 99%, and most preferably from about 85% to about 95% by weight.

One suitable diluent or carrier for use in the compositions of the invention comprises water together with one or more components selected from aliphatic alcohols containing two to four, more preferably two or three, carbon atoms.

An example of topically-acceptable diluent or carrier useful in the present invention is therefore a hydroalcoholic system comprising, by weight of the total composition, from about 1% to about 99% of ethanol, isopropanol, t-butanol or mixtures thereof, and from about 1% to about 99% of water. Preferred is a carrier comprising from about 5% to about 60% by weight of ethanol, isopropanol, or mixtures thereof, and from about 40% to about 95% by weight of water. More preferred is a carrier comprising from about 20% to about 50% by weight of ethanol, isopropanol, or mixtures thereof, and from about 50% to about 80% by weight of water.

Further Anti-Acne Agents

The compositions useful in the present invention contain further anti-acne agents in addition to the one or more compounds of formula (I). It is believed that the compounds of formula (I) have particularly advantageous properties in terms of their anti-acne properties when used in combination with further anti-acne agents.

Preferably, the further anti-acne agent is selected from desquamators, keratolytics, comedolytics and exfoliants. Desquamators, keratolytics, comedolytics and exfoliants aid in the penetration of the active into the skin, and compounds which are capable of serving one or more of these functions are well known in the art. A compound may have one or more of these properties, for example a desquamator may also act as a keratolytic.

The further anti-acne agent is preferably selected from one or more of benzoyl peroxide, resorcinol, resorcinol monoacetate, sulfur, salicylic acid, derivatives of salicylic acid having one or more (C₁ to C₁₂) alkyl and/or (C₁ to C₁₂) alkoxy groups on the aromatic ring (e.g., 5-n-octyl salicylic acid, 5-n-octanoyl salicylic acid and 2-hydroxy-3-methoxybenzoic acid), phenol, cresol, metacresyl acetate, lactic acid, glycolic acid, pyruvic acid, malic acid, urea, N-acetyl cysteine, retinoic acid, retinol, retinyl esters and combinations of retinol and retinyl esters with retinoid boosters. Retinoid boosters are compounds that mimic the effect of retinoic acid on skin by enhancing the conversion of retinol or retinyl esters to retinoic acid. Retinoid boosters may be used singly or as combinations of two or more compounds. Retinoid boosters are described in WO 02/02074, the contents of which are incorporated herein by reference. Specific retinoid boosters include, for example, ceramides, phosphatidyl choline, linoleic acid, 12-hydroxystearic acid and climbazole.

The most preferred further anti-acne agents are selected from one or more of benzoyl peroxide, sulfur and salicylic acid.

These other anti-acne agents are preferably present in the compositions in an amount of from about 0.1% to about 20% by weight, more preferably from about 0.1% to about 10%, and most preferably from about 0.1% to about 5%. Mixtures of these additional anti-acne actives may also be used.

A further group of anti-acne agents that may be included in compositions of the invention, in addition to the above anti-acne agents or instead of the above anti-acne agents, are antibacterials (including antibiotics and antimicrobials), antifungals, antiprotozoals, and antivirals (e.g., benzoyl peroxide, octopirox, erythromycin, triclosan, azelaic acid and its derivatives, phenoxy ethanol and phenoxy propanol, ethyl acetate, clindamycin and meclocycline, chlorhexidine, tetracycline, neomycin, miconazole hydrochloride, octopirox, parachlorometaxylenol, nystatin, tolnaftate, clotrimazole, cetylpyridinium chloride and the like).

Other anti-acne agents include: sebostats such as flavonoids; antipruritic drugs including, for example, topically-acceptable salts of methdilizine and trimeprazine; and bile salts such as scymnol sulfate and its derivatives, deoxycholate, and cholate. The compositions may also comprise pantothenic acid or a pantothenic acid derivative, as described in U.S. Pat. No. 5,612,324, the contents of which are incorporated herein by reference.

Also useful are non-steroidal anti-inflammatory drugs (NSAIDS). The NSAIDS can be selected from the following categories: propionic acid derivatives; acetic acid derivatives; fenamic acid derivatives; biphenylcarboxylic acid derivatives; and oxicams. All of these NSAIDS are fully described in the U.S. Pat. No. 4,985,459 to Sunshine et al., issued Jan. 15, 1991, incorporated by reference herein. Most preferred are the propionic NSAIDS including but not limited to aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Also useful are the steroidal anti-inflammatory drugs including hydrocortisone and the like.

The further anti-acne agent may be a single anti-acne agent or a combination of more than one anti-acne agents.

Surfactants

The compositions useful in the methods of the present invention can optionally comprise one or more surfactants in an amount up to 30% by weight. The surfactants are preferably present at a level from about 0.1% to about 10%, more preferably from about 0.2% to about 5%, and most preferably from about 0.2% to about 2.5% by weight of the composition. Suitable surfactants include, but are not limited to, nonionic surfactants such as glyceryl carboxylates (e.g., glyceryl stearate), polyalkylene glycol ethers of fatty alcohols (e.g., PPG-15 stearyl ether), anionic surfactants such as taurates and alkyl sulfates and amphoteric surfactants such as cetyl betaine. Nonlimiting examples of these surfactants include isoceteth-20, steareth-21, sodium methyl cocoyl taurate, sodium methyl oleoyl taurate, and sodium lauryl sulfate (see for example U.S. Pat. No. 4,800,197 (Kowcz et al.), which is incorporated herein by reference in its entirety). Examples of a broad variety of additional surfactants useful herein are described in McCutcheon's, Detergents and Emulsifiers, North American Edition (1986), published by Allured Publishing Corporation, which is incorporated herein by reference in its entirety.

Preservatives

The compositions of the invention may optionally comprise one or more preservatives for maintaining the antimicrobial integrity of the compositions. Although the compounds of formula (I) may act to some extent as antimicrobial preservatives, the compositions of the invention preferably comprise one or more preservative compounds which are not compounds of formula (I). Preferably, the composition comprises from 0.001% to 5% by weight of the composition, of the one or more preservative compounds which are not compounds of formula (I). Examples of such compounds are triclosan, benzoic acid, benzoic acid salts (e.g., sodium benzoate), benzyl alcohol, hexamidine, o-phenyl phenol, phenoxyethanol, dichlorobenzyl alcohol, iodopropynyl butylcarbamate and preferably C1 to C4 alkyl 4-hydroxybenzoates (parabens), such as methyl paraben and propyl paraben.

Humectants

Another optional component of the compositions useful in the instant invention is at least one humectant (which term includes moisturizers and skin conditioners). A variety of these materials can be employed and each can be present at a level of from about 0.1% to about 20%, more preferably from about 1% to about 10% and most preferably from about 2% to about 5% by weight of the composition. These materials include urea; guanidine; glycolic acid and glycolate salts (e.g. ammonium and quaternary alkyl ammonium); lactic acid and lactate salts (e.g. ammonium and quaternary alkyl ammonium); extracts of natural products, such as extracts of Pyrus malus and aloe vera in any of its variety of forms (e.g., aloe vera gel); polyhydroxy alcohols such as sorbitol, glycerol, hexanetriol, propylene glycol, hexylene glycol and the like; polyethylene glycol; sugars and starches; sugar and starch derivatives (e.g., alkoxylated glucose); hyaluronic acid; lactamide monoethanolamine; acetamide monoethanolamine; and mixtures thereof.

Gel-Forming Materials

Another optional component of the compositions useful in the methods of the instant invention is a gel-forming material. Examples of such a material are carboxylic copolymers (acrylic acid copolymers). Most preferred is Carbomer 1342 (available as Carbopol 1342 from B. F. Goodrich). These polymers are more fully described in U.S. Pat. No. 4,509,949 (Huang et al) and U.S. Pat. No. 2,798,053 (Brown), both of which are incorporated herein by reference in their entirety. Also useful are the acrylate/alkyl acrylate crosspolymers such as Acrylates/C10-C30 Alkyl Acrylate Crosspolymer (available as Pemulen TR-1 and Pemulen TR-2 from Goodrich).

These polymers may be present in the compositions in an amount of from about 0.025% to about 0.75%, preferably from about 0.05% to about 0.25% and most preferably from about 0.075% to about 0.18% by weight of the composition.

Emollients

The compositions useful in the methods of the present invention can also optionally comprise at least one emollient. Examples of suitable emollients include, but are not limited to, volatile and non-volatile silicone oils, highly branched hydrocarbons, hydrogenated castor oil and non-polar carboxylic acid and alcohol esters (e.g., dibutyl adipate), and mixtures thereof. Emollients useful in the instant invention are further described in U.S. Pat. No. 4,919,934 (Deckner et al.), which is incorporated herein by reference in its entirety.

The emollients are typically present in the compositions in an amount of from about 1% to about 50%, preferably from about 1% to about 25%, and more preferably from about 1% to about 10% by weight of the compositions.

pH

A wide variety of acids, bases, and buffers can be utilized to adjust and/or maintain the pH of the compositions useful in the instant invention. Although triethanolamine is preferred, other nonlimiting examples of materials useful for adjusting and/or maintaining the pH include sodium carbonate, sodium hydroxide, hydrochloric acid, phosphoric acid, sodium hydrogen phosphate, sodium dihydrogen phosphate, citric acid, and the like.

Other Optional Components

In addition to the required components of the compositions useful in the present invention, a variety of optional components can also be incorporated. Preferred optional components include antioxidants (e.g., BHT), fragrances, clays (e.g., bentonite), silicones, and pigments. These optional materials may be used singly, or two or more of each type of materials may be used (for example, a composition may include two or more different clays). These optional components may be used in admixture e.g., a composition may contain a preservative, a fragrance and a clay.

Specific examples of optional components, some of which are also mentioned hereinafter, are alkyl alcohols containing from 12 to 24 carbon atoms, alkyl carboxylic acids containing from 12 to 24 carbon atoms, polyvinyl pyrrolidone, polyethylene glycol, mineral oil, polysorbates, nonionic surfactants, sorbitol, methyl cellulose, propylene glycol esters, zinc salts, titanium dioxide and mixtures thereof.

A variety of additional ingredients can be incorporated into the compositions useful in the present invention. Non-limiting examples of these additional ingredients include other vitamins and derivatives thereof (e.g., ascorbic acid, vitamin E, tocopheryl acetate, and the like); thickening agents (e.g. polyacrylamide and C13-14 isoparaffin and laureth-7, available as Sepigel from Seppic Corporation); resins; gums; cationic polymers and thickeners (e.g., cationic guar gum derivatives such as guar hydroxypropyltrimonium chloride and hydroxypropyl guar hydroxypropyltrimonium chloride, available as the Jaguar C series from Rhone-Poulenc; copolymers of acrylamide and a cationic acrylate (available as Salcare SC92 from Allied Colloid); emulsifiers; polymers for aiding the film-forming properties and substantivity of the composition (such as a copolymer of eicosene and vinyl pyrrolidone, an example of which is available from GAF Chemical Corporation as Ganex V-220@R); skin penetration aids such as DMSO, 1-dodecyl-azacycloheptan-2-one (available as Azone from the Upjohn Co.) and the like; artificial tanning agents such as dihydroxyacetone and the like; skin bleaching (or lightening) agents including but not limited to hydroquinone, ascorbic acid, kojic acid and sodium metabisulfite; chelators and sequestrants; and aesthetic components such as colorings, essential oils, skin sensates, astringents, skin soothing agents, skin healing agents and the like, nonlimiting examples of these aesthetic components include clove oil, menthol, camphor, eucalyptus oil, eugenol, menthyl lactate, witch hazel distillate, allantoin, bisabalol, dipotassium glycyrrhizinate and the like. Further optional components include antistatic agents (e.g., distearyldimonium chloride and oxidised polyethylene), viscosity controlling agents (e.g., sodium chloride), bittering agents to inhibit against ingestion (e.g., denatonium benzoate) and components to protect against degradation by light (e.g., benzophenone-2).

Product Forms

Product types suitable for the compositions of the invention include: skin products such as creams, lotions, ointments, sunscreens, anti-aging formulations, sunless tanners; colour cosmetics, including foundations and moisturisers; perfumes; hair treatments including shampoos, conditioners, mousses and gels; personal wash products including soap bars and shower gels; and shaving preparations. The products may take any shape or form. They may be liquids (preferably emulsions), gels, sticks, aerosols, mousses, skin patches, wiping articles, pads, pastes or powders.

Delivery Methods for the Compositions

The compositions useful in the invention can be delivered directly from the package or container for use by the user of the product (e.g., by application via the user's hand or fingers) or from a a variety of delivery devices. The following are two nonlimiting examples.

Medicated Cleansing Pads

The compositions useful herein can be incorporated into a medicated cleansing pad. Preferably these pads comprise from about 50% to about 75% by weight of one or more layers of nonwoven fabric material and from about 20% to about 75% by weight (on dry solids basis) of a water soluble polymeric resin. Examples of pads are described in U.S. Pat. No. 4,891,228, (Thaman et al.) and U.S. Pat. No. 4,891,227 (Thaman et al.), both of which are incorporated by reference herein in their entirety.

Dispensing Devices

The compositions useful herein can also be incorporated into and delivered from a soft-tipped or flexible dispensing device. These devices are useful for the controlled delivery of the compositions to the skin surface and have the advantage that the treatment composition itself never need be directly handled by the user. Nonlimiting examples of these devices comprise a fluid container including a mouth, an applicator, means for holding the applicator in the mouth of the container, and a normally closed pressure-responsive valve for permitting the flow of fluid from the container to the applicator upon the application of pressure to the valve. The valve can include a diaphragm formed from an elastically fluid impermeable material with a plurality of non-intersecting arcuate slits therein, where each slit has a base which is intersected by at least one other slit, and where each slit is out of intersecting relation with its own base, and wherein there is a means for disposing the valve in the container inside of the applicator. Examples of these applicator devices are described in U.S. Pat. No. 4,693,623 (Schwartzman), U.S. Pat. No. 4,620,648 (Schwartzman), U.S. Pat. No. 3,669,323 (Harker et al.), U.S. Pat. No. 3,418,055 (Schwartzman) and U.S. Pat. No. 3,410,645 (Schwartzman), all of which are incorporated herein by reference in their entirety.

Methods for Treating Acne

The present invention relates to a method for treating acne in humans. Such a method comprises topically applying to the skin an effective amount of a composition of the invention. The term “effective amount”, as used herein, means an amount sufficient to provide an anti-acne benefit. The composition can be continually applied at appropriate intervals, preferably about once or twice a day until the acne subsides.

The method preferably involves topical, local application to the area of the skin affected by acne or susceptible to acne, for example the face, forehead, neck and back. Therefore, compositions of the invention are preferably suitable for topical application to the face and/or forehead and/or neck and/or back.

The invention is particularly useful for treating acne vulgaris. The organisms implicated in acne vulgaris are typically Propionibacterium species, especially P. acnes.

The following non-limiting examples illustrate the invention. In the examples and throughout the specification, all percentages are percentages by weight based on the total weight of the composition unless otherwise indicated.

### EXAMPLES

### Example 1

In vitro model systems, reproducing the biotransformation of glucose by Propionibacterium spp., or fatty acid by Corynebacterium spp., to propionic acid, were used to demonstrate the antibacterial properties of 4-hexylresorcinol, an active ingredient according to the invention, and 2,4,4′-trichloro-2′-hydroxydiphenyl ether (triclosan), a benchmark antibacterial agent. The propionibacterial model involved the use of 7 ml glass vials, to which were added 6.0 ml synthetic medium (see below), supplemented with glucose substrate (2.0 mg/ml). The corynebacterial model involved the use of 250 ml baffled shake flasks, to which were added 30.0 ml synthetic medium (see below), supplemented with fatty acid substrate (2.0 mg/ml pentadecanoic acid). To each vial or flask (other than controls) was also added one of the indicated test materials, from a 1.0-10.0% (w/v) stock solution in 50% (v/v) ethanol. Each assay was inoculated with fresh bacterial biomass (Propionibacterium acnes G63 [skin isolate], P. avidum NCTC 11864 or Corynebacterium sp. NCIMB 40928), pre-grown for 24-48 h, under aerobic (corynebacteria) or anaerobic (propionibacteria) conditions, in TSBT (see below), to give starting optical densities (A₅₉₀) of ˜1.0. Following inoculation, the vials and flasks were incubated aerobically (corynebacteria) or anaerobically (propionibacteria) at 35° C., with agitation (120 rpm), for 24 h. After this time, culture viability was determined by total viable count (TVC) analysis on ACX plates (see below), following serial dilution in quarter-strength Ringers solution. Also, at the end of each experiment, the extent of glucose or fatty acid biotransformation to propionic acid was measured by capillary gas chromatography (GC) (see below), and the dose response data used to calculate, for each active ingredient, a BIC so value (50% biotransformation inhibition concentration). The mode of action (bacteriostatic or bactericidal) at this level was elucidated by reference to culture viability data, which was also used to determine the MBC (minimum bactericidal concentration), defined as the minimum level of each active effecting a >3 log₁₀ ml^{31 1} reduction in viability (Tables 1 & 2). Finally, the data for P. acnes G63 and P. avidum NCTC 11864 were combined to generate mean and range anti-propionibacterial values (Table 1).

Composition of synthetic medium (g/l):

KH₂PO₄ (1.6); (NH₄)₂HPO₄ (5.0); Na₂SO₄ (0.38); KNO₃ (0.1); NaHCO₃ (1.0); sodium thioglycolate (1.0); sodium pyruvate (1.0); yeast nitrogen base (Difco) (3.35); MgCl₂.6H₂O (0.5).

Composition of TSBT (g/l):

Tryptone soya broth (Merck) (30.0); yeast extract (Beta Lab) (10.0); Tween 80™ (5.0).

Composition of ACX (g/l):

blood agar base no. 2 (Oxoid) (39.5); yeast extract (Beta Lab) (3.0); glucose (2.0); Tween 80 ™ (5.0); defibrinated horse blood (50 ml/l).

Methodology for GC determination of propionic acid:

1.0 ml aliquots from each assay vial were transferred into sampling tubes, an internal standard (1.0 mg/ml caproic acid) added to each tube, and the culture medium acidified (pH˜2) by the addition of HCl. Liquid-liquid extraction was then carried out using 2 vol (2 ml) ethyl acetate, with organic and aqueous phases being resolved by centrifugation (2000 rpm, 2 min). ˜0.75 ml of each organic (upper) phase was then transferred to a fresh sampling tube prior to analysis on a Perkin Elmer 8000 (Series 2) GC fitted with a 15 m×0.32 mm (internal diameter) FFAP fused silica capillary column (film thickness 0.25 μm) (Quadrex). Metabolite levels were quantified by comparison of peak areas with known amounts of both internal (caproic acid) and externally-run (propionic acid) standards. GC running conditions as indicated: injector (split/splitless), 300° C.; detector (flame ionisation), 300° C.; oven, 80° C. (2 min), 80-155° C. (7.5° C./min), 155° C. (1 min); injection volume, 1.0 μl.

TABLE 1
Anti-propionibacterial properties
of 4-hexylresorcinol and triclosan

| | ¹BIC₅₀ | | ³MBC |
|---|---|---|---|
| Test Material | (ppm) | ²BIC₅₀ MoA(s) | (ppm) |
| 4-Hexylresorcinol | 42 | Bacteriostatic/ | 50-100 |
| | | Bactericidal | |
| Triclosan | 39 | Bacteriostatic/ | 50-100 |
| | | Bactericidal | |

¹BIC₅₀, mean concentration at 50% inhibition of glucose biotransformation to propionic acid, by P. acnes G63 and P. avidum NCTC 11864;
²Mode(s) of action at BIC₅₀ levels for P. acnes G63 and P. avidum NCTC 11864;
³Range of minimum bactericidal levels for P. acnes G63 and P. avidum NCTC 11864.

TABLE 2
Anti-corynebacterial properties of 4-hexylresorcinol and triclosan

| | ¹BIC₅₀ | | ³MBC |
|---|---|---|---|
| Test Material | (ppm) | ²BIC₅₀ MoA(s) | (ppm) |
| 4-Hexylresorcinol | 118 | Bacteriostatic | >500 |
| Triclosan | 20 | Bacteriostatic | 50 |

¹BIC₅₀, concentration at 50% inhibition of pentadecanoic acid biotransformation to propionic acid, by Corynebacterium sp. NCIMB 40928;
²Mode of action at BIC₅₀ level for Corynebacterium sp. NCIMB 40928;
³Minimum bactericidal levels for Corynebacterium sp. NCIMB 40928.

The results show that 4-hexylresorcinol has anti-propionibacterial properties that are as good as those of Triclosan, and that 4-hexylresorcinol exhibits far greater selectivity than Triclosan for Propionibacterium spp. compared to other bacteria normally present on the skin.

### Examples 2 to 5

The following are examples of topical compositions which are prepared by combining the following components utilizing conventional mixing techniques.

| | % Weight | | | |
|---|---|---|---|---|
| Ingredients | Ex. 2 | Ex. 3 | Ex. 4 | Ex. 5 |
| Deionized Water | qs 100 | qs 100 | qs 100 | qs 100 |
| Ethanol (SD 40B Alcohol) | 35.0 | 35.0 | 20.0 | 35.0 |
| Salicylic Acid | 2.0 | 2.0 | 0.5 | 2.0 |
| 4-Hexylresorcinol | 3.0 | 3.0 | 1.0 | 3.0 |
| Glycerol | 2.0 | 2.0 | — | — |
| Menthol | — | — | 0.05 | — |
| Witch Hazel Distillate | — | — | 5.0 | — |
| Na Methyl Cocoyl Taurate | — | — | 1.0 | — |
| Isoceteth-20 | — | — | — | 2.0 |
| PPG-15 stearyl ether | 0.1 | 0.1 | | |
| Quaternium-22 | — | — | 1.0 | — |
| Disodium EDTA | 0.005 | 0.005 | 0.005 | 0.005 |
| Triethanolamine, 99% | 0-1.0 | 0-1.0 | — | 0-1.0 |

### Example 6

A gel composition is prepared by combining the following components utilizing conventional mixing techniques.

| Ingredients | % Weight |
|---|---|
| Deionized Water | Qs 100 |
| Ethanol (SD 40B Alcohol) | 40.0 |
| 4-Hexylresorcinol | 5.0 |
| Salicylic Acid | 2.0 |
| Polyacrylamide thickener | 4.0 |

### Example 7

A gel composition is prepared by combining the following components utilizing conventional mixing techniques.

| Ingredients | % Weight |
|---|---|
| Deionized Water | Qs 100 |
| Ethanol (SD 40B Alcohol) | 40.0 |
| 4-Hexylresorcinol | 5.0 |
| Climbazole | 1.0 |
| Copolymer of Acrylamide and | 3.0 |
| Cationic Acrylate¹ | |
| Glyceryl Stearate | 0.05 |
| Menthol | 0.05 |
| Disodium EDTA | 0.05 |
| Glycerol | 2.00 |

¹Available as Salcare SC92 from Allied Colloids.

1. An anti-acne composition for topical application comprising:
(i) from 0.01% to 20% by weight of a compound of formula (I) 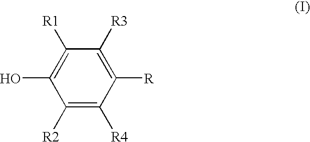
wherein:
R1, R2, R3 and R4 are the same or different and are selected from H, OH, and OCH₃; and
R is alkyl containing from 1 to 20 carbon atoms;
the compound of formula (I) optionally being in the form of an ester with a carboxylic acid comprising an alkyl group containing from 1 to 6 carbon atoms;
(ii) between 0.1% and 20% by weight of at least one further anti-acne agent;
(iii) from 0% to 30% by weight of at least one surfactant; and
(iv) a topically acceptable diluent or carrier.

2. Composition as claimed in claim 1, wherein the further anti-acne agent is selected from desquamators, keratolytics, comedolytics and exfoliants.

3. Composition as claimed in claim 2, wherein the further anti-acne agent is selected from one or more of benzoyl peroxide, resorcinol, resorcinol monoacetate, sulfur, salicylic acid, derivatives of salicylic acid having one or more (C₁ to C₁₂) alkyl and/or (C₁ to C₁₂) alkoxy groups on the aromatic ring, phenol, cresol, metacresyl acetate, lactic acid, glycolic acid, pyruvic acid, malic acid, urea, N-acetyl cysteine, retinoic acid, retinol, retinyl esters and combinations of retinol and retinyl esters with retinoid boosters.

4. Composition as claimed in claim 3, wherein the further anti-acne agent is selected from one or more of benzoyl peroxide, sulfur and salicylic acid.

5. Composition as claimed in claim 1, wherein the compound of formula (I) has the formula (II) 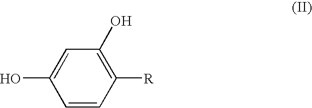
wherein R is as defined in claim 1.

6. Composition as claimed in claim 1, wherein R is alkyl containing 4 to 14 carbon atoms.

7. Composition as claimed in claim 1, wherein R is alkyl containing 5 to 8 carbon atoms.

8. Composition as claimed in claim 1, wherein the compound of formula (I) is 4-hexylresorcinol.

9. Composition as claimed in claim 1, wherein the surfactant is present in an amount of from 0.1% to 10% by weight.

10. Composition as claimed in claim 1, wherein the cosmetically acceptable diluent or carrier comprises water together with one or more components selected from aliphatic alcohols containing two or three carbon atoms.

11. Composition as claimed in claim 1, which comprises one or more of a fragrance, a clay, a surfactant, a gel-forming material, a silicone, an emollient, an antioxidant, a humectant and a pigment.

12. A method of using a composition as defined in claim 1 in the manufacture of a medicament for the treatment of acne.

13. Method of treating acne in humans which comprises topically applying to the skin of a human suffering from acne a composition of claim 1.

14. The method as claimed in claim 12, wherein the acne is caused by Propionobacterium acnes.

15. The method as claimed in claim 13, wherein the acne is caused by propionobacterium acnes.
